(19) United States
(12) Patent Application Publication
(10) Pub. No.: US 2026/0060998 A1
(43) Pub. Date: Mar. 5, 2026

(54) BACTERIAL CELLULOSE FOR TREATING OR PREVENTING AIRWAY DISEASES

(71) Applicant: Chao-Cheng CHEN, Taipei City (TW)

(72) Inventors: Qiong WANG, Taipei (TW); Hang Ming DONG, Taipei (TW); Qi YU, Taipei (TW); Chao-Cheng CHEN, Taipei City (TW); Si Han ZHENG, Taipei (TW)

(21) Appl. No.: 19/319,809

(22) Filed: Sep. 5, 2025

(30) Foreign Application Priority Data
Sep. 5, 2024 (CN) 202411246552.0

(51) Int. Cl. A61K 31/717; A61P 11/06

(52) U.S. Cl. CPC A61K 31/717; A61P 11/06

(57) ABSTRACT

The present disclosure provides a use of a bacterial cellulose in treating and preventing airway diseases, especially the use in the treatment and prevention of inflammatory airway diseases associated with an impairment in airway epithelial cells such as asthma.

### BACKGROUND

### 1. Technical Field

The present disclosure relates to a use of a bacterial cellulose in treating or preventing airway diseases, especially to a use of the bacterial cellulose in treating or preventing an inflammatory airway disease worsen by an impairment in airway epithelial cells.

### 2. Description of Associated Art

Asthma is a chronic airway disease characterized in airway inflammation, airway hyper responsiveness, increased mucous secretion, etc. Airway epithelial cells play an important role in the pathogenesis of asthma. Epithelial cells are known to release a large amount of inflammatory factors (IL-25, IL-33, TSLP, etc.) when contacting environmental allergens or pathogens The release of these epithelial inflammatory factors (also referred to as alarmins) activates a large amount of immune cells such as Th2 and ILC2 cells, finally resulting in eosinophilic airway inflammation, and also activates mast cells directly to result in airway inflammation and airway hyper responsiveness.

Destruction of epithelial cells can be found in all phenotypes of asthma, the epithelial cells of a patient with asthma are impaired, which is characterized in epithelial cells peeled off, reduced expression of intercellular adhesion molecules such as claudin (ZO-1, Claudins, occludin, TJP2) and cohesin (β-catenin and E-cadherin), resulting in impaired barrier function and increased permeability for external pathogens and allergens. On the other hand, the impaired epithelial cells and reduced expression of intercellular claudins and cadherins enhance the possibility of epithelial-mesenchymal transition (EMT) of airway epithelial cells and worsen the airway remodeling in the patient with asthma, thereby resulting in exacerbation of asthma.

The pathogenesis including the destruction of epithelial cells and impairment of the barrier function has been found extensively in other airway diseases. However, the current treatment of airway diseases is not aimed at repairing destructed epithelial cells and has no efficacy in repairing the destructed epithelial cells, and therefore, the airway diseases cannot be completely cured and will reoccur when environmental allergens and pathogens contact the epithelial cells again.

In the past decade, the rapid development of nanotechnology promotes the creation of many materials with good potential in application. Various functional nanomaterials with anti-inflammatory and anti-oxidizing effects have been developed, such as metal oxide nanoparticles, carbon nanomaterials, and noble metal nanoparticles, and have been reported to find use in treating diseases including stroke, sepsis, inflammatory bowel diseases, neurodegenerative diseases, diabetes, acute kidney injury, and acute liver injury.

Additionally, cellulose is one of natural macromolecular materials of the highest abundance and nanomaterials of the greatest interests, and in recent years, bacterial cellulose (BC), a highly crystalline linear glucose biopolymer generated from bacterial fermentation, has focused more attention. BC possesses the unique physical and chemical properties of high elasticity modulus, high specific surface area, low density, non-abrasive, ease to surface functionality, high purity of chemical components, high crystalline degree, high degree of polymerization (2000-8000), and good biocompatibility and biodegradability, etc. Bacterial cellulose has been applied in wound dressing, vascular tissue engineering, and bone tissue regeneration and is demonstrated to be well biocompatible.

However, a nanomaterial is still rarely used in airway diseases. Due to the special anatomical structure and immune environment of lung, it is greatly meaningful to develop a nanomaterial with a smaller particle size, a higher degree of evenness, a better in vivo stability, milder components, and a better biocompatibility.

### SUMMARY

Given various disadvantages of prior art described above, the present disclosure provides a use of a composition in the manufacture of a medicament for treating or preventing airway diseases, wherein the composition comprises a bacterial cellulose formed of β-1-4-glucan, and the bacterial cellulose has a diameter of 15-35 nm and a length of 100-3000 nm.

The present disclosure further provides a method of treating or preventing airway diseases in a subject in need thereof, wherein the method comprises administering a therapeutically effective amount of a composition comprising a bacterial cellulose formed of β-1-4-glucan, and the bacterial cellulose has a diameter of 15-35 nm and a length of 100-3000 nm.

The present disclosure also provides a composition comprising a bacterial cellulose formed of β-1-4-glucan for use in treating or preventing airway diseases, wherein the composition comprises a bacterial cellulose with a diameter of 15-35 nm and a length of 100-3000 nm.

In an embodiment, the bacterial cellulose has a length-to-diameter ratio of 2.85-86.

In an embodiment, an amount of the bacterial cellulose in the composition is 0.2 wt% to 1.2 wt%.

In an embodiment, the composition is administered to a subject in need thereof at a dose of 0.1-0.5 mg of the bacterial cellulose per kg of body weight 1-4 times per day.

In an embodiment, the composition is administered to the subject via a nasal administration or an inhalation, preferably, the nasal administration comprises a drop or spray form, and the inhalation comprises a nebulizer or dry powder form.

In an embodiment, the composition further comprises a carrier, the bacterial cellulose is dispersed in the carrier, and the carrier is selected from water, physiological saline, buffer, and Ringer's solution.

In an embodiment, the composition further comprises at least one selected from the group consisting of a flavoring agent, a dispersing agent, a wetting agent, a lubricant, a thickening agent, a stabilizer, a preservative, an antioxidant, an antimicrobial agent, and a coloring agent.

In an embodiment, the bacterial cellulose is generated by at least one bacterium selected from the group consisting of Gluconacetobacter, Acetobacter, Rhizobium, Sarcina, Pseudomonas, Achromobacter, Alcaligenes, Enterobacter, Azotobacter, and Agrobacterium; preferably, the bacterial cellulose is generated by bacteria of Gluconacetobacter and/or Acetobacter.

In an embodiment, the airway disease is selected from asthma, chronic obstructive pulmonary disease, acute respiratory distress syndrome, bronchitis, allergic rhinitis, chronic cough, and pulmonary alveolitis; preferably, the airway disease is asthma.

Specifically, the bacterial cellulose used in present disclosure has a good biocompatibility in lung and a function of maintaining the barrier homeostasis of airway epithelium. Therefore, the bacterial cellulose can be used in lung, and the bacterial cellulose can effectively recover the impaired epithelial barrier function and reduce inflammatory syndromes of asthma, thereby shortages in current treatment of airway diseases can be covered.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present disclosure can be more fully understood by reading the following descriptions of the embodiments, with reference made to the accompanying drawings.

FIGS. 1A-1C are graphs showing the results of the animal toxicity experiment performed on the bacterial cellulose of the present disclosure. FIG. 1A shows the dosage regimen; FIG. 1B shows no statistical difference in weight change during the toxicity experiment between groups of mice; and FIG. 1C shows the (H&E and PAS) staining images of lung pathological sections of mice in each group.

FIGS. 2A-2I present graphs showing the results of the experiment performed on the bacterial cellulose of the present disclosure to reduce the airway injury and inflammatory reaction in animal asthma models. FIG. 2A shows a dosing regimen for producing mice asthma models by induction with HDM; FIG. 2B shows the (H&E and PAS) staining images of lung pathological sections of mice in each group; FIGS. 2C-2G show the expression levels of inflammatory factors IL-13 (FIG. 2C), IL-5 (FIG. 2D), IL-25 (FIG. 2E), IL-33 (FIG. 2F), and TSLP (FIG. 2G) in bronchoalveolar lavage fluid (BALF) of mice in each group; FIG. 2H shows the immunofluorescent staining images for claudin (ZO-1) and cohesin (β-catenin and E-cadherin) of lung tissue epithelial cells of mice in each group, with the scale being 50 μm; and FIG. 2I shows the histogram obtained after quantification of the immunofluorescent images in FIG. 2H. For the data of ZO-1, β-catenin and E-cadherin in each group, the data from Control group, HDM group, HDM+2 mg/kg BC group, and HDM+4 mg/kg BC group are displayed from left to right. Values are expressed as mean±standard deviation and analyzed by one-way ANOVA and Tukey multiple-range test. * represents P<0.05, ** represents P<0.01, *** represents P<0.001, and ns represents no significant difference.

FIGS. 3A-3C present graphs showing the results of the experiment performed on the bacterial cellulose of the present disclosure to reduce the airway injury in cell asthma models. FIG. 3A shows the experimental results graph of CCK8 cytotoxicity test; FIG. 3B shows the expression levels of β-catenin and E-cadherin in HDM-induced asthma cell models with bacterial cellulose at different concentrations tested with Western blotting method, of which the bottom panel shows results after quantification, for the data from E-cadherin and β-catenin in each group, the data from the Control group, HDM group, 0.4 g/mL group, 0.8 g/mL group, 1.6 g/mL group, and 3.2 g/mL group are shown from left to right; and FIG. 3C shows the immunofluorescent staining images for claudin (ZO-1) and cohesin (E-cadherin) of cells in each group. One-way ANOVA and Tukey multiple-range test are conducted to analyze. * represents P<0.05, ** represents P<0.01, *** represents P<0.001, and ns represents no significant difference.

FIGS. 4 and 5 are electron microscopic images of the bacterial cellulose of the present disclosure. FIG. 4 shows that the bacterial cellulose of the present disclosure has an uniform size and a good dispersing property without tangles or aggregation; and FIG. 5 further shows the size of the bacterial cellulose of the present disclosure which has a diameter of 15-35 nm and a length of 100-3000 nm.

### DETAILED DESCRIPTION OF THE PREFERRED EMBODIMENTS

The embodiments of the present disclosure will be illustrated by the following embodiments, and those skilled in the art can easily understand the advantages and benefits of the present disclosure from the content described in this specification. The present disclosure can be practiced or applied by other different implementations, and different modifications and alternations can be made to the details of the present specification based on different views and applications without departing from the spirit described in the present disclosure.

In the present disclosure, when referred to as “comprise/comprising”, “include/including” or “have/has/had” particular elements, other elements such as members, components, structures, regions, portions, devices, systems, steps or connection can be additionally comprised, unless otherwise specified.

The singular form “a/an” and “the” also comprises the plural form, unless otherwise indicated clearly, and the “or” can be interchangeably used with “and/or” herein.

The numerical value ranges recited in the present disclosure are inclusive and combinable, any numerical values falling in the numerical value ranges can be used as the upper or the lower limit to derive subranges thereof; for example, the numerical value range of “diameter of 15-35 nm” can be understood as any subrange including 15 nm as the lower limit and 35 nm as the upper limit, e.g., a subrange such as 15-30 nm, 16-35 nm, and 22-28 nm, etc. ; in addition, a value falling in any range (such as between the upper and lower limits) recited in the present disclosure should be included in the range of the present disclosure.

The bacterial cellulose of the present disclosure refers the cellulose generated by bacteria, e.g., generated by bacterial fermentation culturing, which is formed from D-glucose connected with each other in β(1→4) glucoside and belongs to β-1-4-glucan. Unlike the plant cellulose, the bacterial cellulose has a higher purity.

In one embodiment, the bacterial cellulose has a diameter of 15-35 nm, e.g., about 15 nm, about 16 nm, about 17 nm, about 18 nm, about 19 nm, about 20 nm, about 21 nm, about 22 nm, about 23 nm, about 24 nm, about 25 nm, about 26 nm, about 27 nm, about 28 nm, about 29 nm, about 30 nm, about 31 nm, about 32 nm, about 33 nm, about 34 nm, or about 35 nm. In one embodiment, the bacterial cellulose has a length of 100-3000 nm, e.g., about 100 nm, about 150 nm, about 200 nm, about 250 nm, about 300 nm, about 350 nm, about 400 nm, about 450 nm, about 500 nm, about 550 nm, about 600 nm, about 650 nm, about 700 nm, about 750 nm, about 800 nm, about 850 nm, about 900 nm, about 950 nm, about 1000 nm, about 1050 nm, about 1100 nm, about 1150 nm, about 1200 nm, about 1250 nm, about 1300 nm, about 1350 nm, about 1400 nm, about 1450 nm, about 1500 nm, about 1550 nm, about 1600 nm, about 1650 nm, about 1700 nm, about 1750 nm, about 1800 nm, about 1850 nm, about 1900 nm, about 1950 nm, about 2000 nm, about 2050 nm, about 2100 nm, about 2150 nm, about 2200 nm, about 2250 nm, about 2300 nm, about 2350 nm, about 2400 nm, about 2450 nm, about 2500 nm, about 2550 nm, about 2600 nm, about 2650 nm, about 2700 nm, about 2750 nm, about 2800 nm, about 2850 nm, about 2900 nm, about 2950 nm, or about 3000 nm. In some embodiments of the present disclosure, the diameter refers to an average diameter, the length refers to an average length, i.e., the bacterial cellulose has an average diameter between 15 nm and 35 nm and an average length between 100 nm and 3000 nm. In at least one embodiment of the present disclosure, the bacterial cellulose has a diameter preferably between 15 nm and 25 nm, and a length preferably between 100 nm and 1000 nm, more preferably between 200 nm and 650 nm. In an embodiment, the bacterial cellulose has a length-to-diameter ratio of 2.85-86, e.g., about 2.85, 3, 4, 5, 10, 15, 20, 25, 30, 35, 40, 45, 50, 55, 60, 65, 70, 75, 80, 85, or 86.

In the present disclosure, the bacterial cellulose formed by β-1-4-glucan can be used in manufacture of a composition for treatment or prevention of airway diseases including administration of the bacterial cellulose formed by β-1-4-glucan to a subject in need thereof. In some embodiments, the composition of the present disclosure can be a pharmaceutical composition but is not limited thereto. In some embodiments of the present disclosure, the airway disease can be asthma, chronic obstructive pulmonary disease, acute respiratory distress syndrome, bronchitis, allergic rhinitis, chronic cough, or pulmonary alveolitis, preferably, the airway disease treated or prevented in the present disclosure is asthma. The composition of the present disclosure can effectively treat or prevent airway diseases, or prevent worsen of the airway diseases.

In one embodiment, a subject in need of treatment or prevention of airway diseases can be administered with the bacterial cellulose formed by β-1-4-glucan or a composition thereof by nasal administration directly, e.g., by administration in a form of drop or spray, but not limited thereto. In another embodiment, a subject in need of treating or preventing airway diseases can be administered with the bacterial cellulose formed by β-1-4-glucan or a composition thereof by direct inhalation, e.g., by oral or nasal inhalation, also for example, by inhalation via a form of nebulizer or dry powder, but not limited thereto.

In one embodiment, the bacterial cellulose formed by β-1-4-glucan or the composition including the same of the present disclosure can be dried, e.g., by freeze-drying to form dry bulky frozen-dried tablets or dry powders and then processing the dray powders into tablets, prior to utilization. In some embodiments of the present disclosure, the bacterial cellulose or the composition including the same can be subjected to at least one process of deep freezing, pumping at low temperature and low pressure, and desorption, allowing the overall article of manufacture to be more stable, difficult to deterioration, and beneficial for storage. In utilization, since the bacterial cellulose fiber has excellent reconstruction properties, a dry article of manufacture (e.g., freeze-dried tablets, dry powders, or tablets) can be easily mixed with a liquid carrier, thereby forming a composition including the liquid carrier and the bacterial cellulose. In some embodiments of the present disclosure, the bacterial cellulose or the composition including the same can be sequentially processed by deep freezing, pumping at low temperature and low pressure, and desorption, however, it should be understood that the order of treatment processes is not limited thereto, which can be appropriately adjusted based on the properties of the produced bacterial cellulose.

In some embodiments, dosage forms commonly used for airway diseases can be formulated by using the composition of the present disclosure, which is convenient for self-administration or for administration to a subject by another one. In some embodiments of the present disclosure, the dosage forms can be, for example, drop or spray administered nasally, or a nebulizer or dry powder for administration by inhalation. in addition to the dry dosage form of dry powder, other dosage forms such as drop, spray, nebulizer includes a liquid carrier, and the liquid carrier can include non-irritating liquid, e.g., water, physiological saline, buffer, and Ringer's solution, but not limited thereto. In the case of administrating the composition to a subject with drop, spray or in a nebulizer, since the composition includes liquid carrier, it is not necessary to mix the bacterial cellulose produced in the present disclosure with a liquid carrier, prior to utilizaiton of the composition of the present disclosure.

In some embodiments of the present disclosure, based on a total weight of the composition, an amount of the bacterial cellulose can be between 0.2 wt% and 1.2 wt%, thereby enhancing the dispersing rate thereof. For example, the fiber is present at an amount of about 0.2 wt%, about 0.3 wt%, about 0.4 wt%, about 0.5 wt%, about 0.6 wt%, about 0.7 wt%, about 0.8 wt%, about 0.9 wt%, about 1.0 wt%, about 1.1 wt%, or about 1.2 wt%. In some embodiments of the present disclosure, the fiber may be not able to provide sufficient hydroxyl groups when the content of the fiber is less than 0.2 wt%, influencing the interfacial tension of a liquid medium to some extent. In some embodiments of the present disclosure, when the content of the fiber is less than 0.2 wt %, the liquid medium and the fiber would aggregate due to the influence of cohesion, thus causing phase separation which is not favorable to mixing.

In some embodiments, the bacterium for producing bacterial cellulose can be at least one selected from Gluconacetobacter, Acetobacter, Rhizobium, Sarcina, Pseudomonas, Achromobacter, Alcaligenes, Enterobacter, Azotobacter, Agrobacterium, or any combination thereof. In some embodiments, the bacterium can be selected from Gluconacetobacter and/or Acetobacter. In some embodiments, the bacterium can be at least one selected from the group consisting of Acetobacterxylinum (or referred to as Gluconacetobacterxylinus), Gluconacetobacterhansenii, and Gluconacetobactersacchari. In some embodiments of the present disclosure, a bacterial species of Gluconacetobacter, especially Acetobacterxylinum, can be chosen to produce the bacterial cellulose, but not limited thereto. In some embodiments of the present disclosure, a single or a plurality of bacterial species can be chosen to produce the bacterial cellulose, and can be modified based on actual requirement without limit.

In order to prepare of the bacterial cellulose of the present disclosure, a container charged with a culturing medium can be prepared firstly, then the single or the plurality of bacterial species is(are) cultured in the container charged with the culturing medium in a manner of setting for 24-96 hours (such as 24, 36, 48, 60, 72, 84, or 96 hours), with the absorbance value (at a wavelength of 620 nm) of the bacterial concentration being controlled in a range from 0.005 to 0.01, e.g., about 0.005, about 0.006, about 0.007, about 0.008, about 0.009, or about 0.01; in some embodiments of the present disclosure, the pH value of the culture solution is controlled in an acidic range, including a pH value between 0.5 and 6.5, such as about 0.5, about 1.0, about 1.5, about 2.0, about 2.5, about 3.0, about 3.5, about 4.0, about 4.5, about 5.0, about 5.5, about 6.0, or about 6.5; in some embodiments of the present disclosure, the concentration of bacteria in the culture solution is controlled in a range from 102 to 105 bacteria/mL, such as about 1×10² bacteria/mL, about 5×102 bacteria/mL, about 1×10³ bacteria/mL, about 5×10³ bacteria/mL, about 1×10⁴ bacteria/mL, about 5×10⁴ bacteria/mL, or about 1×10⁵ bacteria/mL; in some embodiments of the present disclosure, the culturing temperature can be controlled between 25° C. and 30° C., such as about 25° C., about 26° C., about 27° C., about 28° C., about 29° C., or about 30° C. In some embodiments of the present disclosure, the absorbance value of the bacterial concentration in the culture medium described above, pH value of the culture medium, range of the bacterial concentration in the culture medium, culturing temperature, or any combination thereof can be selected to control the bacterial culture of the present disclosure.

As used in the present disclosure, the “static culture” is allowing bacteria to form a laminar fiber membrane in a non-woven manner on the surface (i.e., gas-liquid interface) of the culture medium. In addition, the container used for static culture can be a flat-shaped container with a broad culture region to control oxygen consumption by the bacteria through the less height of the container, thereby achieving the regulation of bacterial cellulose diameter. In some embodiments of the present disclosure, since the network structure formed by fibers at the surface of the formed fiber membrane is more dense and compact than that inside the fiber membrane, it is beneficial for subsequent separation of the interlaced bacterial cellulose through the static culture and culturing condition.

As used in the present disclosure, the “fiber membrane” refers to as a laminar article formed by interlacing a plurality of fibers and having multiple layers of network structure. In some embodiments of the present disclosure, the fiber membrane can have a thickness between 20 μm and 30 μm, such as about 20 μm, about 22 μm, about 24 μm, about 25 μm, about 26 μm, about 28 μm, or about 30 μm. In some embodiments of the present disclosure, the amount of the bacterial cellulose per unit area is between 0.001 g/cm² and 0.002 g/cm², such as about 0.0011 g/cm², about 0.0012 g/cm², about 0.0013 g/cm², about 0.0015 g/cm², about 0.0017 g/cm², about 0.0018 g/cm², or about 0.0019 g/cm². In some embodiments of the present disclosure, the bacterial cellulose in the fiber membrane has a diameter between 15 nm and 100 nm, such as about 15 nm, about 20 nm, about 25 nm, about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, or 100 nm.

In at least one embodiment, the components of the culture medium can include: carbon source, nitrogen source, and gel support. The carbon source can include at least one of sugars or sugar alcohols such as mannitol, glucose, molasses, etc. ; the nitrogen source can include peptone, yeast extract, or a combination thereof; and the gel support can be selected from, e.g., agar, but not limited thereto. In some embodiments of the present disclosure, the culture medium can include agar, carbon source, peptone, and yeast extract, and the weight ratio of the carbon source, the peptone, and the yeast extract can be 5:1:1 to 4:1:1.

In at least one embodiment of the present disclosure, the fiber membrane can be prepared by static fermentation of bacteria of genus Gluconacetobacter in a culture medium containing mannitol, peptone, yeast extract, and agar, and the prepared fiber membrane has a water content higher than 85%, such as a water content higher than 90%, higher than 92%, or higher than 95%.

In at least one embodiment, the bacterial cellulose of the present disclosure is obtained by forming a fiber membrane through bacterial fermentation culture and then processing the fiber membrane. In order to obtain the bacterial cellulose, processing the fiber membrane includes performing a fiber splitting treatment on the fiber membrane, which includes at least one selected from the group consisting of pulverizing homogeneously, swelling with a solvent, and mechanically milling. In at least one embodiment, the fiber splitting treatment includes pulverizing homogeneously, swelling with a solvent, and mechanically milling performed in sequence, but not limited thereto.

As used in the present disclosure, the “homogeneously pulverizing” is performed by mixing the fiber membrane with a solvent, and then pulverizing with a homogenizing device composed of fixed outer blades having shearing forces and rotatable inner blade having a saw-toothed shape, to obtain a dispersion solution.

In at least one embodiment of the present disclosure, into the dispersion solution after the homogenization treatment described above, other additives are added to promote separation of the interlaced bacterial cellulose. The additives can include other additives commonly used in the field, but not limited thereto.

As used in the present disclosure, the “swelling with a solvent” includes allowing a treatment solution to permeate inside the interlaced bacterial cellulose in the dispersion solution, thereby weakening the hydrogen bonding effects between cellulose without performing excess hydrolysis on the bacterial cellulose, and thus being capable of reducing energy consumption during the subsequent mechanical milling. In some embodiments of the present disclosure, with the synergistic effect of the shearing force generated by the mechanical milling, the glucoside bonds in the bacterial cellulose break, achieving the fiber splitting of the bacterial cellulose, which increases the specific surface area of the cellulose and expose more hydroxy groups, thereby increasing the hydrophilicity and biocompatibility of the bacterial cellulose.

In at least one embodiment, the treatment solution can be at least one selected from the group consisting of base solution, inorganic salt solution, and ionic liquid aqueous solution. In some embodiments of the present disclosure, the base for forming the base solution can include at least one selected from the group consisting of potassium hydroxide, sodium hydroxide, and lithium hydroxide; in some embodiments of the present disclosure, the inorganic salt can be at least one selected from the group consisting of urea, zinc chloride, urea sulfide, calcium chloride, and magnesium chloride; in some embodiments of the present disclosure, the ionic liquid can be at least one selected from the group consisting of 1-allyl-3-methylimidazolium chloride ([AMIm]Cl), 1-butyl-3-methylimidazolium chloride ([BMIm]Cl), 1-allyl-3-methylimidazolium acetate ([AMIm]Ac), 1-butyl-3-methylimidazolium acetate ([BMIm]A), lithium chloride/dimethyl sulfoxide (LiCl/DMSO), N-alkylimidazoles, and dialkylimidazoles.

As used in the present disclosure, the “mechanical milling” includes diluting the dispersion solution by adding water, and performing a milling treatment with a horizontal ball miller to achieve fiber splitting on the interlaced bacterial cellulose in the dispersion solution, the size of the bacterial cellulose is determined to a great extent during this process, for example, the size with a diameter of 15-35 nm and a length of 100-3000 nm exemplified in the present disclosure. The bacterial cellulose for mechanical milling includes between about 0.1 wt% and about 5 wt%, such as about 0.1 wt%, about 0.2 wt%, about 0.3 wt%, about 0.4 wt%, about 0.5 wt%, about 0.6 wt%, about 0.7 wt%, about 0.8 wt%, about 0.9 wt%, about 1.0 wt%, about 1.2 wt%, about 1.5 wt%, about 1.8 wt%, 2 wt%, about 2.5 wt%, about 3 wt%, about 3.5 wt%, about 4 wt%, about 4.5 wt%, or about 5 wt% of the total weight of the dispersion solution.

In addition to above, the processing can further include purification. The purification is performed at last, for example, after homogeneously pulverizing, swelling with a solvent, and/or mechanically milling, the dispersion solution is purified, but not limited thereto. The purification can be referenced to known methods, for example, including neutralizing and/or desalting, e.g., separating salts in the dispersion solution out through semi-permeable membrane dialysis, to obtain bacterial cellulose fibers with desired morphology.

In at least one embodiment of the present disclosure, the milled bacterial cellulose has high specific surface area and more significant electrostatic effect, van der Waals force and hydrogen bonding force between cellulose, and may be easy to aggregate. Therefore, in some embodiments, the present disclosure can include additionally treating the milled dispersion with ultrasonication after mechanical milling to collapse the bacterial cellulose aggregates, and thereafter, performing freeze-drying based on the actual requirement.

In some embodiment, the composition of the present disclosure can further include at least one selected from the group consisting of a flavoring agent, a dispersing agent, a wetting agent, a lubricant, a thickening agent, a stabilizer, a preservative, an antioxidant, an antimicrobial agent, and a coloring agent, and there will be no adverse effect between the bacterial cellulose of the present disclosure and each component described above.

In some embodiments of the present disclosure, the bacterial cellulose formed by β-1-4-glucan or the composition including the bacterial cellulose can be administered at the time of, before or after occurrence of symptoms, to achieve the preventing, treating, and alleviating effects. For example, the composition of the present disclosure is administered at a dose of 0.1-0.5 mg, e.g., 0.1 mg, 0.15 mg, 0.2 mg, 0.25 mg, 0.3 mg, 0.35 mg, 0.4 mg, 0.45 mg, or 0.5 mg of the bacterial cellulose per kg of body weight.

In some embodiments of the present disclosure, the bacterial cellulose formed by β-1-4-glucan or the composition including the bacterial cellulose can be administered at the time of or after occurrence of symptoms to alleviate the symptoms. In some embodiments of the present disclosure, the mode of administration can be as described above.

In at least one embodiment of the present disclosure, the bacterial cellulose formed by β-1-4-glucan or the composition including the bacterial cellulose can be administered nasally twice a day with an interval of 1-12 hours, e.g., with an interval of 1 hour, 2 hours, 3 hours, 4 hours, 5 hours, 6 hours, 7 hours, 8 hours, 9 hours, 10 hours, 11hours, or 12 hours, to treat or alleviate the symptoms of airway diseases. In some embodiments of the present disclosure, the bacterial cellulose formed by β-1-4-glucan or the composition including the bacterial cellulose can be administered before meal, after meal, before sleep, or at the onset time of symptoms. In one embodiment, the bacterial cellulose formed by β-1-4-glucan or the composition including the bacterial cellulose can be administered nasally three times a day with intervals of 4-8 hours. Optionally, more than 3 times, e.g., 4 times also can be administered to gain more obvious alleviation of symptoms.

### EXAMPLES

The present disclosure will be illustrated in detail by specific Examples below which should not to be considered to limit the scope of the present disclosure.

Test Example 1: animal experiments

1.1 Establishment of an animal toxicity experimental model

12 SPF-grade female C57BL/6 mice were fed in the SPF-grade animal room at the 3rd Floor, Experimental Animal Research Center, Nanfang Hospital. Mice were randomized to 3 groups (Control group; 2 mg/kg BC group; and 4 mg/kg BC group), 4 mice/group. Starting from Day 0, after anesthesia with isoflurane, the Control group was dosed with 20 μL PBS daily by nasal drop, while the 2 mg/kg BC group and the 4 mg/kg BC group were dosed with the BC composition (which was obtained by dispersing the bacterial cellulose having a diameter of 15-35 nm and a length of 150-1000 nm in PBS, and the bacterial cellulose was obtained from the cellulose produced by Acetobacterxylinum after processing and milling according to the method described above) corresponding to a concentration of 20μL by nasal drop respectively, to reach doses of 2 mg and 4 mg bacterial cellulose per kg body weight respectively. Dosing was continued for 14 days, and samples were collected within 24 hours after dosing at Day 14.

### 1.2 Preparation of Asthma Animal Models

20 SPF-grade female C57BL/6 mice were fed in the SPF-grade animal room at the 3rd Floor, Experimental Animal Research Center, Nanfang Hospital. Mice were randomized into 4 groups (Control group; HDM group; HDM+2 mg/kg BC group; HDM+4 mg/kg BC group), 5 mice/group. The regimen for animal test could refer to previous studies, for example, Huang H, Qiao Y, Chu L, et al. Up-regulation of HSP90α in HDM-induced asthma causes pyroptosis of airway epithelial cells by activating the cGAS-STING-ER stress pathway. Int Immunopharmacol.

### 1.2.1 Sensitization of HDM Asthma Model Animals

60 μL PBS and 40 μL HDM were used to formulate 100 μL 4000U HDM (ALUTARD Mites Allergens Alk 100000SQ-U/mL), and mice in the HDM group, HDM+2 mg/kg BC group, and the HDM+4 mg/kg BC group were injected intraperitoneally with 100 μL 4000U HDM on Day 0 and Day 7 since animal modeling.

### 1.2.2 Stimulation and Treatment in HDM Asthma Animal Model p 6μL PBS and 4 μL HDM were used to formulate 10 μL 400U HDM (ALUTARD Mites Allergens Alk 100000SQ-U/mL), from Day 8 to Day 21 of modeling, mice in the HDM group, the HDM+2 mg/kg BC group, and the HDM+4 mg/kg BC group were administered daily after isoflurane inhalation anesthesia, and at 1 hours after dosing of HDM, mice in the HDM+2 mg/kg BC group and the HDM+4 mg/kg BC group were given the BC composition (which was obtained by dispersing the bacterial cellulose having a diameter of 15-35 nm and a length of 150-1000 nm in PBS, and the bacterial cellulose was obtained from the cellulose produced by Acetobacterxylinum after processing and milling according to the method described above) corresponding to a concentration of 20 μL, to reach doses of 2 mg and 4 mg bacterial cellulose per kg body weight, respectively. Samples were collected within 24 hours after dosing at Day 21.

### 1.3 ELISA Assay of Bronchoalveolar lavage fluid

The left lungs of mice in each group collected in Test Example 1.2.2 were ligated, and the right lung tissues of the mice were perfused in three portions with 1.5 mL of physiological saline, the mice bronchoalveolar lavage fluid were collected and tested for the expression levels of IL-4, IL-5, IL-13, IL-25, IL-33, and TSLP by using correspondent ELISA detection kits (Gelatins), and the brightness was detected using an ELISA reader.

### 1.4 HE Staining of Mice Lung Tissues

The left lung tissues of mice in each group collected in Examples 1.1 and 1.2.2 were fixed with paraformaldehyde, embedded in paraffin, sliced continuously, and dewaxed and rehydrated, thereafter were stained with hematoxylin and eosin (Bciosharp), mounted, and observed under a microscope.

### 1.5 PAS Staining of Mice Lung Tissues

The left lung tissues of mice in each group collected in Examples 1.1 and 1.2.2 were fixed with paraformaldehyde, embedded in paraffin, sliced continuously, and dewaxed and rehydrated, thereafter were stained with a glycogen PAS staining kit (Solarbio), mounted, and observed under a microscope.

### 1.6 Immunofluorescent Staining of Mice Lung Tissues

The left lung tissues of mice in each group collected in Test Example 1.2.2 were fixed with paraformaldehyde, embedded in paraffin, sliced continuously, and dewaxed and rehydrated, thereafter the tissue sections were antigen-repaired, mounted, incubated with a primary antibody against airway epithelial barrier-related proteins (β-catenin, E-cadherin and ZO-1) (protein tech) at 4° C. overnight, then a secondary antibody (Alexa Fluor Plus 594) was added and incubated at room temperature for 1 hour, then were stained with DAPI (Beyotime Biotechnology) for 5 minutes, mounted with anti-fluorescence quencher, and observed under a fluorescence microscope.

### 1.7 Experimental Results

Many studies about the use of BC in treatment of diseases have demonstrated the good biocompatibility and safety of BC, but there lacks adequate studies about the use of BC in lung, and therefore, since the special anatomical structure and immune environment of lung, in order to confirm if BC can be used safely in lung, the inventors set two doses, 2 mg/kg and 4 mg/kg respectively, as described in Test Example 1.1, to investigate if the use of BC in lung would cause damage, in reference to the maximum gavage dose in previous studies about the orally acute toxicity experiments of BC.

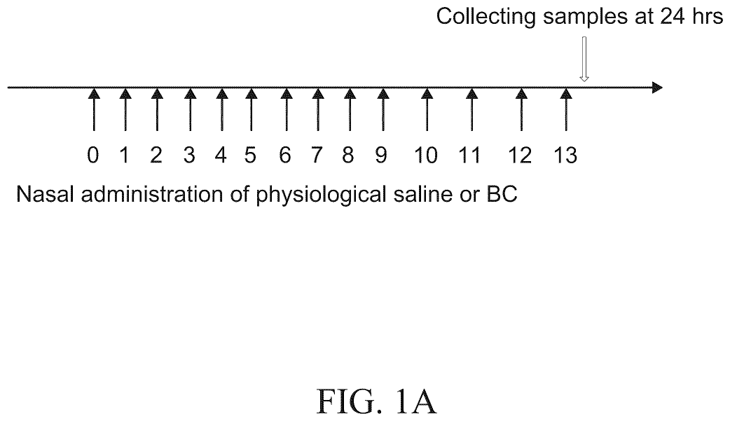

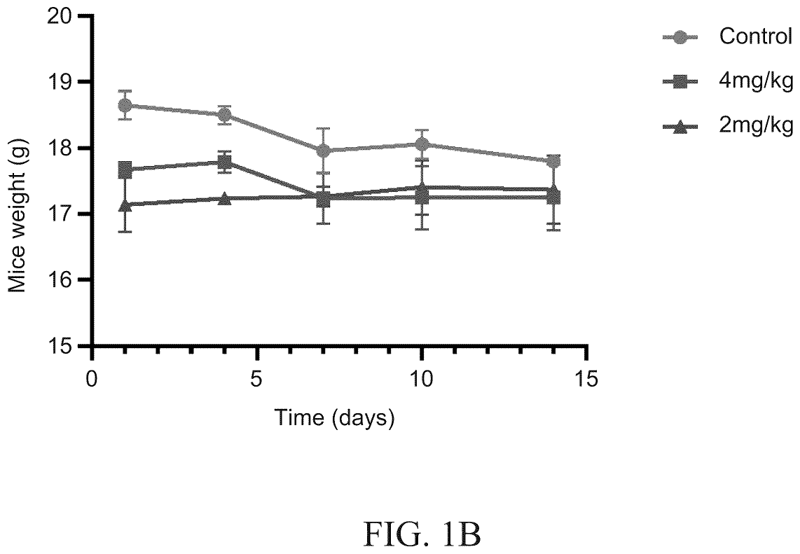

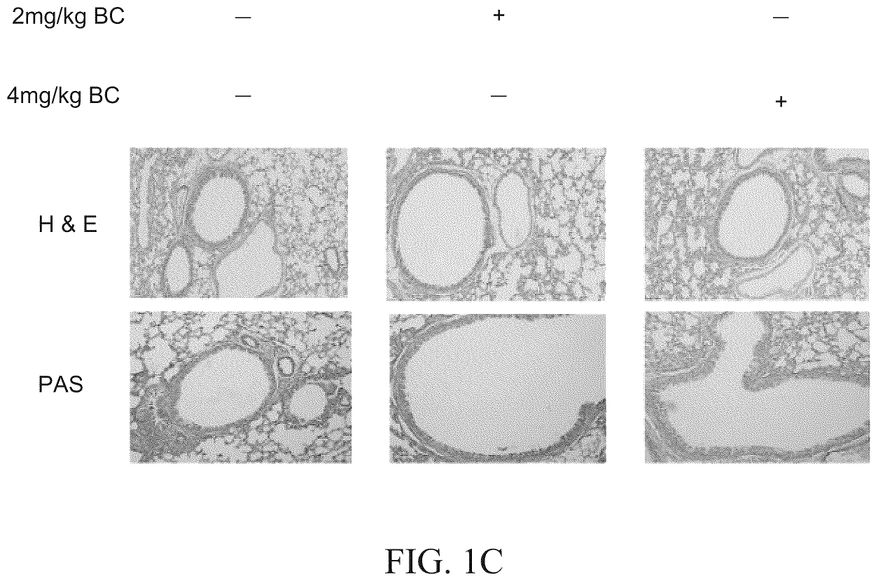

Following the dosing regimen shown in FIG. 1A, mice were administered with different concentrations of BC in a nasal drop manner for consecutive 14 days, and samples were collected within 24 hours of the last dose. Changes in weight of mice during 14 days were recorded (FIG. 1B, n=4), showing no statistic difference in weight changes during 14 days between groups. In the pathological sections of lung tissues obtained as in Test Examples 1.4 and 1.5 (FIG. 1C, n=4, 200× amplification), no obvious destructive proliferation inflammatory cell infiltration in pulmonary mesenchyme and no obvious damage in airway was seen from the HE staining, and no goblet cell hyperplasia or increased mucous secretion was seen from the PAS glycogen staining. The experimental results above showed that the intrapulmonary administration of BC at the doses of 2 mg/kg and 4 mg/kg by nasal drop could not cause death, cachexia, and acute lung injury in mice, and the intrapulmonary administration of BC via nasal drop would therefore be feasible.

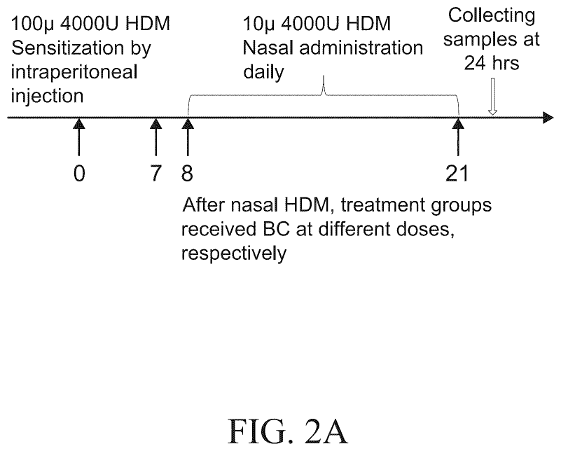

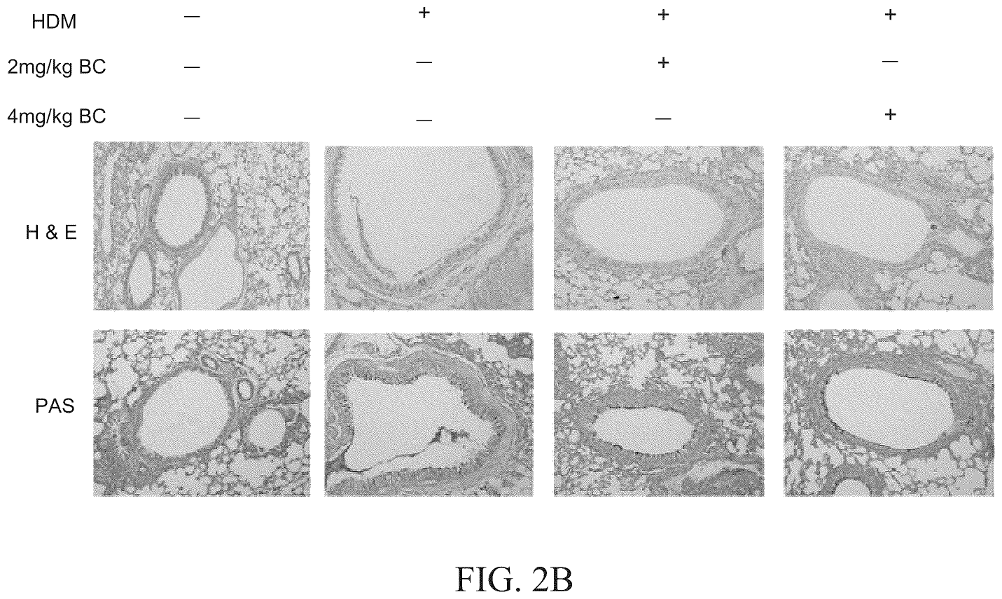

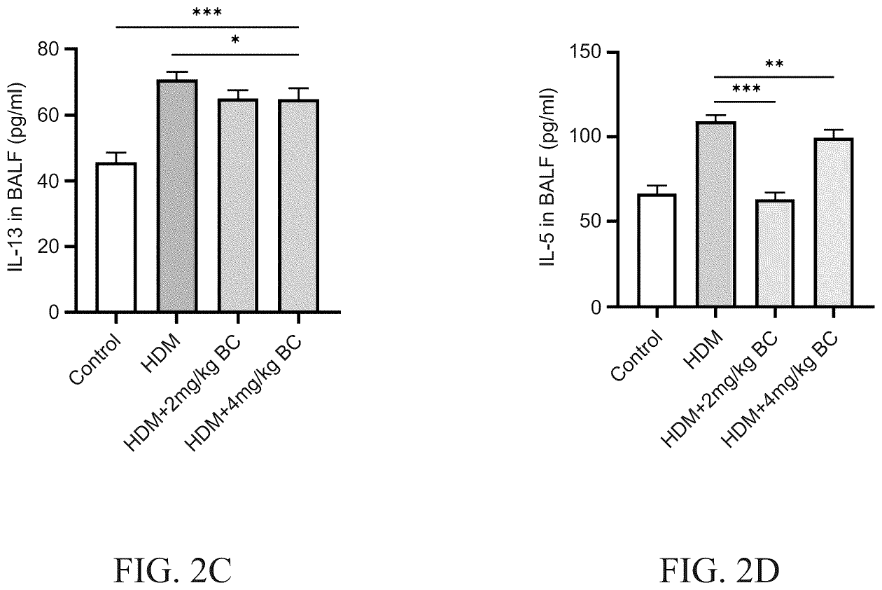

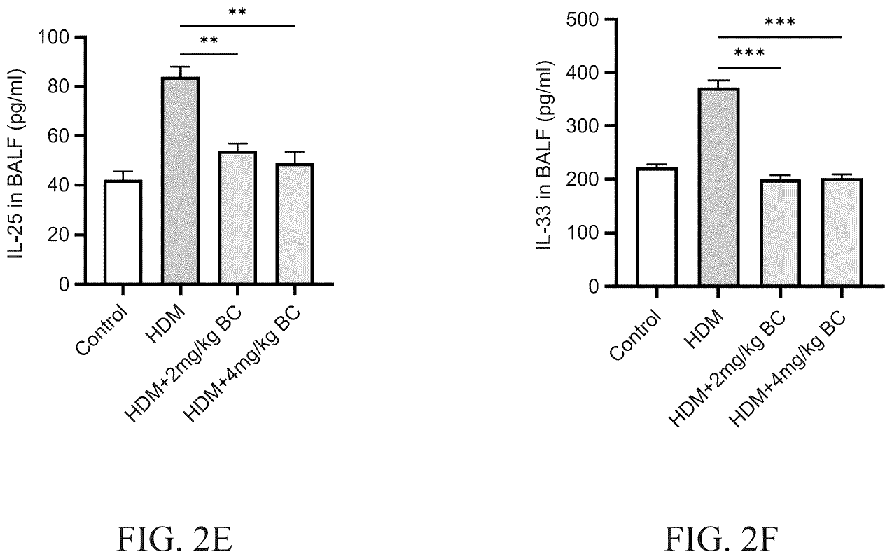

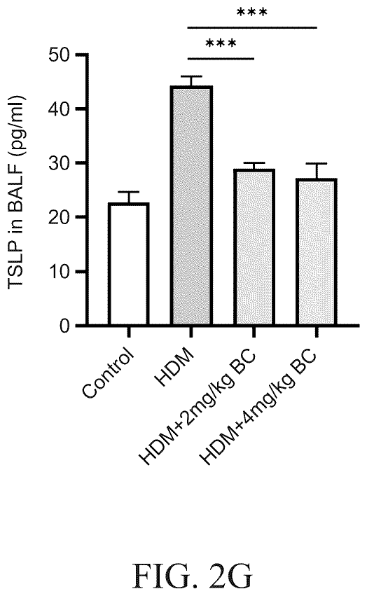

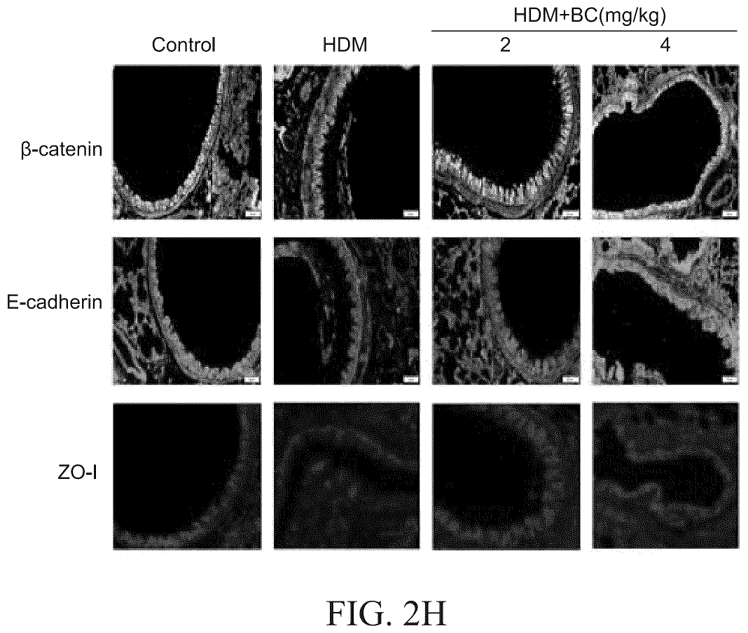

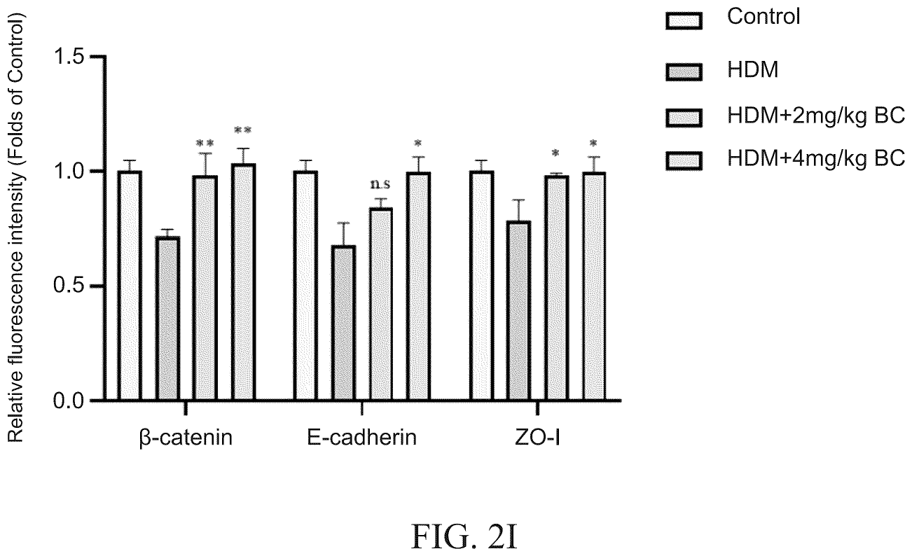

After confirming the safety and feasibility of the intrapulmonary administration of BC at the two doses, HDM-induced mice asthma models were established with the regimens showed in Test Example 1.2 and in FIG. 2A, and tests were performed on the obtained mice lung tissue with the methods of Test Examples 1.4 and 1.5 above. Among these, the He-stained sections (FIG. 2B) showed reduced airway injury in both BC-treated groups with different doses (i.e., the HDM+2 mg/kg BC group and the HDM+4 mg/kg BC group), and significant improvement in airway epithelial continuity and manifestation of airway epithelial cells damage/shedding in the treated groups, in relative to the modeling group (i.e., the HDM group). It also could be seed in the PAS-stained sections (FIG. 2B), mice in the BC-treated groups has decreased mucous secretion and decreased goblet cell hyperplasia, compared to the modeling mice. In other words, the staining of pathological tissue sections showed that, in the HDM-induced asthma model mice, the intrapulmonary administration of BC by nasal drop did reduce the manifestation of asthma-affected airway epithelial injury and decrease the mucous secretion.

The ELISA assay on mice bronchoalveolar lavage fluids (BALFs) as described in Test Example 1.3 above showed that, in both BC-treated groups with different doses (i.e., the HDM+2 mg/kg BC group and the HDM+4 mg/kg BC group), the expression levels of asthma type II inflammatory markers IL-13 (FIG. 2C) and IL-5 (FIG. 2D) were reduced compared to the HDM group with statistic differences, and the expression level of IL-5 in the HDM+2 mg/kg BC group even recovered to that of the Control group. In addition, it was surprisingly found that alarmins associated with epithelial cells, including IL-25, IL-33 and TSLP, in Bc-treated groups with different doses were recovered to the levels in the Control group (FIGS. 2E-2G). That is, ELISA assay on inflammatory factors in BALFs showed that the intrapulmonary administration of BC via nasal drop could reduce asthma airway inflammation in the HDM-induced asthma model mice.

To further confirm if the impaired airway epithelial cell barrier function would be ameliorated after BC treatment, the expression profiles of claudins and cohesins (E-cadherin, ZO-1, β-catenin) on airway epithelial cells in mice lung tissue sections were tested by inventors using the immunofluorescence as described in Test Example 1.6. In the HDM+4 mg/kg BC group, the expression levels of claudins and cohesins on airway epithelial cells recovered to the normal levels (FIGS. 2H and 2I). Otherwise, in the HDM+2 mg/kg BC group, the expression level of each protein was improved compared to that in the HDM group. It was confirmed again by aforementioned that the intrapulmonary BC administration via nasal drop could effectively decrease epithelial impairment and inflammatory factor secretion in the HDM-induced asthma models.

This Test Example has confirmed that in model mice, the administration of BC at the doses of 2 mg/kg and 4 mg/kg has good safety profile and desired effect of reducing airway injury caused by asthma. According to the general conversion method in the arr, the dose in mice should be about 9.01 times greater than that in human (based on a body weight of 60 kg), and it can be expected that a dose about 0.1-0.5 mg per kg body weight should be appropriate when the bacterial cellulose of the present disclosure is administered to an adult human of 60 kg weight.

### Test Example 2: Cell Experiments

### 2.1 Establishment and Treatment of Cell Asthma Models

BEAS-2B cells were cultured with DMEM/10% FBS medium and stimulated with 800U HDM (ALUTARD Mites Allergens Alk 100000SQ-U/ml) for 48 hours for asthma modeling. Among these, the Control group was not stimulated with HDM, the HDM group was stimulated with 800U HDM for 48 hours; and the HDM+0.4 g/mL BC group, the HDM+0.8 g/mL BC group, the HDM+1.6 g/mL BC group and the HDM+3.2 g/mL BC group were treated with the combination of 800U HDM and 0.8gBC, 800U HDM and 1.6gBC, 800U HDM and 3.2 g BC, and 800U HDM and 6.4 g BC, respectively, for 48 hours. The BC was the bacterial cellulose having a diameter of 15-35 nm and a length of 150-1000 nm, which was obtained from the cellulose produced by Acetobacterxylinum after treatment and milling according to the method described above.

### 2.2 CCK8 Cytotoxicity Test Experiment

BEAS-2B cells were seeded in a 96-well plate at a seeding density of 1×10⁴ cells/well, cultured to a cell density of 60%, then the DMEM/10% FBS medium was changed to DMEM medium, and stimulated with BC at different concentrations (0 mg/L, 3.28 mg/L, 6.56 mg/L, 13.12 mg/L and 26.24 mg/L of BC in PBS, and the BC was the bacterial cellulose having a diameter of 15-35 nm and a length of 150-1000 nm, which was obtained from the cellulose produced by Acetobacterxylinum after treatment and milling according to the method described above) for 48 hours. After culturing at 37° C. and 5% CO₂ for 48 h, the medium in each well was replaced with a mixture of the cell count kit 8 (CCK-8) reagent and DMEM medium at a volume ratio of 1:10. Then after culturing in a cell incubator in dark for 1 hours 30 minutes, the absorbance at 450 nm was measured using a Multimode Reader and the cell viability was calculated according to the following Equation: Cell viability=[Test well OD value −Background OD value]/[Control well OD value−Background OD value]×100%.

### 2.3 Western Blotting

A protease inhibitor and a protease phosphate inhibitor at a ratio of 1:100 were added into a high strength RIPA to extract the whole-cell protein from cells in each group of Test Example 2.1. The proteins were isolated through 8% and 12% SDS-PAGE gel and translated onto a PVDF membrane. The PVDF membrane was blocked with a rapid blocking solution at room temperature for 8 min, incubated with the primary antibodies against β-catenin and E-cadherin (Protein Tech) in a refrigerator at 4° C. overnight, then labeled with a near-infrared DyLight fluorescent secondary antibody, and imaged with LI-COR Odyssey infrared fluorescent imager.

### Cell Immunofluorescence

Cells in each group of Test Example 2.1 was fixed with 4% paraformaldehyde at room temperature for 10 min, subjected to membranolysis with 0.1% Triton-X100 for 10 min, and blocked with 5% BSA at room temperature for 1 hour, then primary antibodies against ZO-1 and E-cadherin (CST) were added and the mixture were incubated in a refrigerator at 4° C. overnight, thereafter a secondary antibody (Alexa Fluor Plus 594) was added and incubated at room temperature for 1 hour, the mixture was diluted with DAPI (Beyotime Biotechnology) at 1:1 ratio and stained at room temperature for 2 min, and imaged under a confocal microscopy.

### 2.5 Experiment Results

The animal experiments in Test Example1 above showed that the use of BC in asthma animal models could reduce symptoms of asthma and ameliorate injury of asthma airway epithelium. The inventors also observed that airway epithelial cells could be the main target cells for BC. Therefore, the following experiments were performed to confirm if BC improved symptoms of asthma by acting on airway epithelial cells. Firstly, cells of human bronchial epithelial cell line (BEAS-2B) were chosen to establish asthma models by HDM stimulation, with the details of the experiments as described in Test Example 2.1 above.

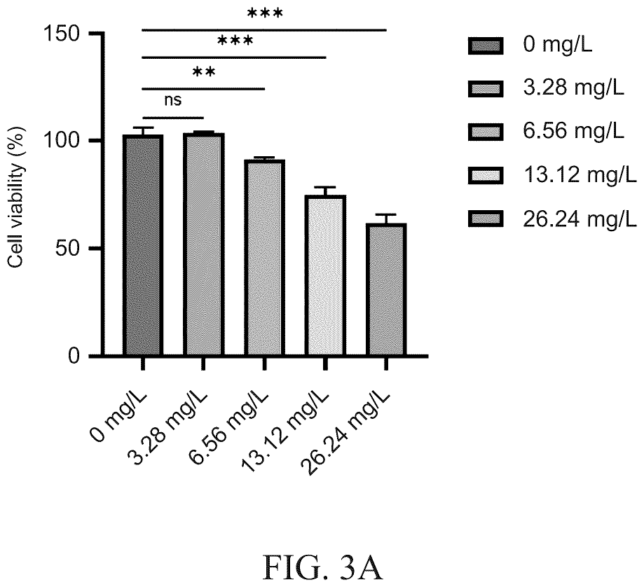

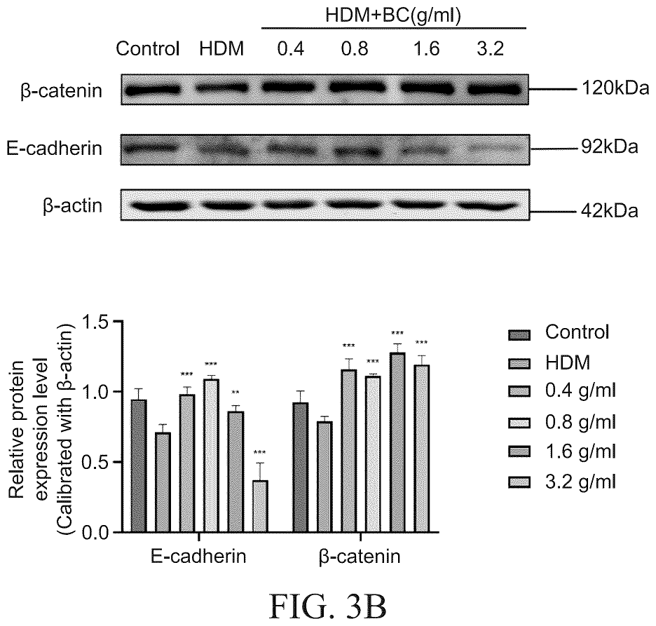

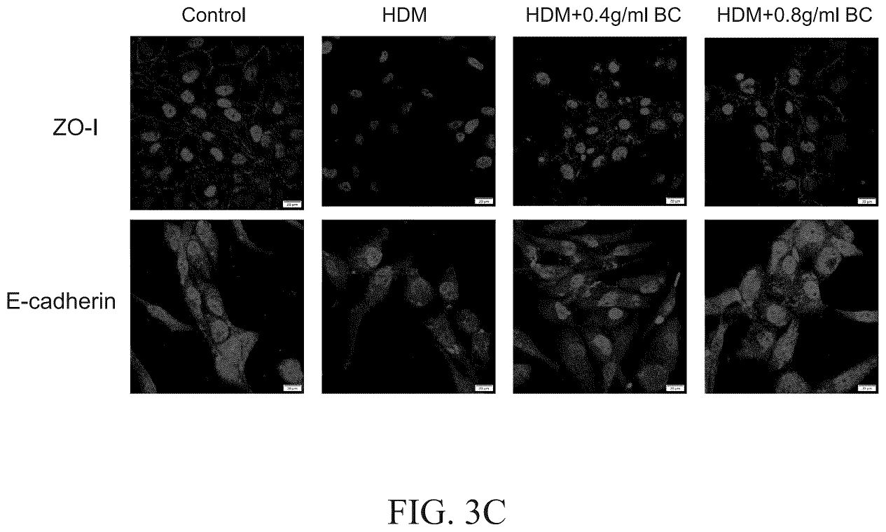

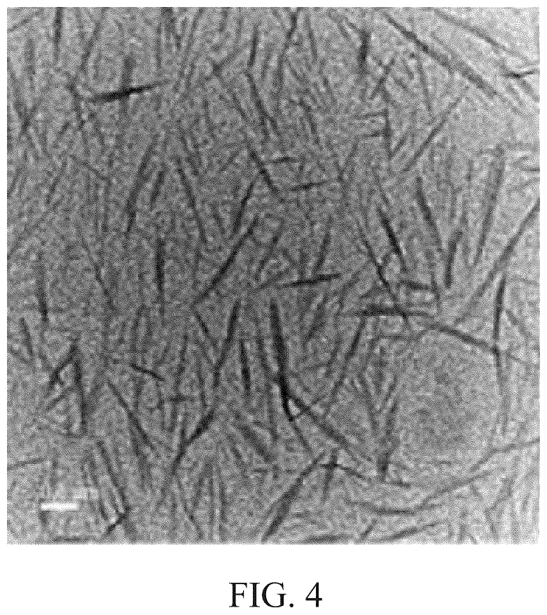

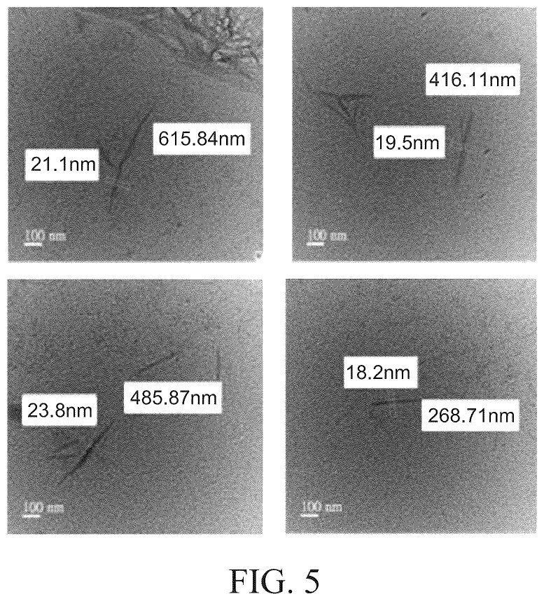

Thereafter, in order to select an appropriate stimulating concentration and to confirm cytotoxicity, the inventors used the CCK8 cytotoxicity test experiment described in Test Example 2.2 above to screen the appropriate concentration. As shown in FIG. 3A, BC at the concentration of 3.28 mg/L could not cause apoptosis of airway epithelial cells.

To further select an appropriate treatment concentration, the BC at concentrations of 0.4 g/mL, 0.8 g/mL, 1.6 g/mL and 3.2 g/mL in combination with 800U HDM were chosen to co-treat the asthma cell model prepared in Test Example 2.1, and the Western blotting as in Test Example 2.3 above was performed to identify the effects of □ BC at different concentrations within a concentration gradient on the expression levels of representative proteins of claudins and cohesins (β-catenin and E-cadherin). As shown in FIGS. 3B, 0.4 g/mL and 0.8 g/mL BC had good effects in amelioration of HDM-stimulated epithelial cell injury.

To further identify the ability of BC at different concentrations in amelioration of impaired epithelial barrier function, the cell immunofluorescence experiment described in Test Example 2.4 above was performed to identify the expression profiles of claudins and cohesin (ZO-1, E-cadherin) on cells in different groups (FIG. 3C). It can be seen from the results that the BC at the concentration of 0.8 g/ml has a better amelioration effect on the injury caused by HDM stimulation, suggesting with an appropriate treatment concentration, a better treatment effect was achieved with a higher concentration.

It can be seed from the Test Examples above, the bacterial cellulose of the present disclosure has a good safety, can ameliorate epithelial cell injury to promote the recovery of epithelial continuity of airway epithelial cells, decrease cell shedding, decrease airway mucous secretion, decrease goblet cell hyperplasia and reduce inflammatory reactions, thereby impaired epithelial barrier can be repaired to achieve the effect of treating or preventing airway diseases, especially airway diseases (e.g., asthma) associated with impairment of epithelial cells. Therefore, the bacterial cellulose of the present disclosure has a practical application prospect.

The examples described above are provided for the purpose of illustration only and not for limiting the present disclosure. Modifications and alternations can be made to the examples described above by one skilled in the art without departing from the spirit and scope of the present disclosure. Therefore, the claimed range of the present disclosure is defined by the claims attached, and should be encompassed in the technical solutions of the present disclosure as long as it has no impact on the results and implementation of the present disclosure.

What is claimed is:

1. A method of treating or preventing airway diseases in a subject in need thereof, wherein the method comprises administering a therapeutically effective amount of a composition comprising a bacterial cellulose formed of β-1-4-glucan, and the bacterial cellulose has a diameter of 15-35 nm and a length of 100-3000 nm.

2. The method of claim 1, wherein the bacterial cellulose has a length-to-diameter ratio of 2.85-86.

3. The method of claim 1, wherein an amount of the bacterial cellulose in the composition is 0.2 wt% to 1.2 wt%.

4. The method of claim 1, wherein the composition is administered to a subject in need thereof at a dose of 0.1-0.5 mg of the bacterial cellulose per kg of body weight 1-4 times per day.

5. The method of claim 4, wherein the composition is administered to the subject via a nasal administration or an inhalation, the nasal administration comprises a drop or spray form, and the inhalation comprises a nebulizer or dry powder form.

6. The method of claim 1, wherein the composition further comprises a carrier, the bacterial cellulose is dispersed in the carrier, and the carrier is selected from water, physiological saline, buffer, and Ringer's solution.

7. The method of claim 1, wherein the composition further comprises at least one selected from the group consisting of a flavoring agent, a dispersing agent, a wetting agent, a lubricant, a thickening agent, a stabilizer, a preservative, an antioxidant, an antimicrobial agent, and a coloring agent.

8. The method of claim 1, wherein the bacterial cellulose is generated by at least one bacterium selected from the group consisting of Gluconacetobacter, Acetobacter, Rhizobium, Sarcina, Pseudomonas, Achromobacter, Alcaligenes, Enterobacter, Azotobacter, and Agrobacterium.

9. The method of claim 1, wherein the airway disease is selected from asthma, chronic obstructive pulmonary disease, acute respiratory distress syndrome, bronchitis, allergic rhinitis, chronic cough, and pulmonary alveolitis.

10. The method of claim 9, wherein the airway disease is asthma.
